(19) United States
(12) Patent Application Publication
(10) Pub. No.: US 2006/0292612 A1
(43) Pub. Date: Dec. 28, 2006

(54) Method for the quality control of functionalized surfaces

(75) Inventor: Matthias Scheffler, Hirschberg (DE)

Correspondence Address: ROTHWELL, FIGG, ERNST & MANBECK, P.C., 1425 K STREET, N.W., SUITE 800, WASHINGTON, DC, 20005, US

(73) Assignee: febit biotech GmbH, Heidelberg (DE)

(21) Appl. No.: 11/452,422

(22) Filed: Jun. 14, 2006

(30) Foreign Application Priority Data
Jun. 15, 2005 (DE) 10 2005 027 667.9

(51) Int. Cl. C40B 30/06; C40B 40/08; C12Q 1/68; G01N 33/53; C12M 1/34

(52) U.S. Cl. 435/6; 435/7.1; 435/287.2

(57) ABSTRACT

The invention relates to a method which can be used to test firstly a reaction and secondly the distribution of functional groups on a surface. This method is employed in a situation that demands documentation or testing of a distribution of surface functionalization, which is as homogeneous as possible.

### DESCRIPTION

The invention relates to a method which can be used to test firstly a reaction and secondly the distribution of functional groups on a surface. This method is employed in a situation that demands documentation or testing of a distribution of surface functionalization, which is as homogeneous as possible. Said functionalization is carried out using a coating method suitable for the surface material and coating molecules. The method of the invention is used in particular for quality control in the preparation of reaction supports for biochips, gene chips and microarrays.

The preparation of reaction supports or arrays firstly comprises functionalization of the support, wherein functional groups are usually applied to the surface in a coating process. The functionalized surfaces are then available in a next processing step for further coating with corresponding biologically or chemically functional materials and, respectively, molecules. Examples of said biologically or chemically functional materials and, respectively, molecules are nucleic acids or nucleic acid analogs such as DNA, RNA, PNA, LNA, oligonucleotides (independently of the direction of synthesis), saccharides and carbohydrates, peptides, proteins and further mixed forms, but also derivatives of combinatorial chemistry or linker molecules, and building blocks for the synthesis of such molecules.

One problem here, however, is the fact that it is not possible to determine the quality of the functionalized surface with sufficient accuracy.

The present invention relates to a method for testing functionalized groups on a surface,
(a) providing a support comprising a surface,
(b) coating at least part of the surface of said support with first functional coating molecules, using first coating molecules which carry a reversibly bound label,
(c) detecting the existence and/or density or/and distribution of the labels on the surface,
(d) removing the labels and
(e) where appropriate, coating the support with further functional coating molecules, which react with functional groups of said first coating molecules, said functional groups having preferably been uncovered due to removal of the labeling groups.

The invention further relates to a reagent, in particular for carrying out the abovementioned method, containing at least one first functional coating molecule having a reversibly bound label and at least one further functional coating molecule capable of reacting with functional groups of said first coating molecule, said functional groups having preferably been uncovered due to removal of the labeling groups.

The supports may be functionalized with customary reagents, for example with silanization reagents for glass supports. According to the invention, the molecules used for functionalization carry a label or a plurality of labels which can be detected by suitable methods. Accordingly, the label may be analyzed using absorption, emission, radioactivity, plasmon resonance, light diffraction, light scattering, ellipsometry, electron diffraction or electrical signals etc. Preference is given to optically measurable markers such as fluorophores. Preference is given to silanes having at least one, for example one or two, functional groups to react with biological substances, and at least one label. Particular preference is given to functionalization reagents containing a silane group for binding to glass surfaces and a building block for nucleic acid synthesis, for example a phosphoramidite group.

The invention is employed in particular in processes comprising a step-by-step synthesis of receptors, preferably of a plurality of different receptors on a single support, from a plurality of synthesis building blocks. The support may be any support, for example a support having a planar or structured surface.

The support is preferably a microfluidic support, in particular a support having closed channels on whose surface receptor synthesis takes place. Preferred examples of supports are described in WO 00/13018 and WO 00/13017.

A suitable reaction procedure must enable the label to be removed from the coating molecules selectively and, in particular, location-specifically, for example by way of chemical and/or photochemical methods. The location-specific uncovering of the reaction center allows the independent synthesis of functional molecules, for example probes (independently of the sequence and/or type of probes, for example oligonucleotides, peptides, etc.). Particular preference is given to molecules disclosed in the application PCT/EP03/14826 “Verfahren zum validierten Aufbau von Arrays” [Method for the validated construction of arrays], in particular types I, II, VI and VII. Furthermore, preference is given to two-stage protective groups, in particular modified trityl groups as described in WO 03/004510. Reference is explicitly made to the contents of these documents.

After analyzing the data, the signals coming from the labeling groups are used for assessing the homogeneity of surface occupation. Subsequently, the labeling groups are removed by chemical reagents and/or irradiation in order to synthesize functional molecules.

The invention provided functionalized supports which have measurable surface characteristics and which can be used as starting materials for constructing reaction supports and arrays.

Special exemplary embodiments based on silanes and fluorescent dyes:

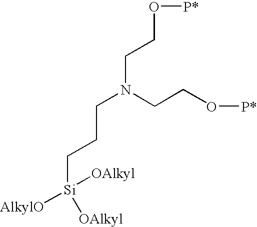

P* [protective group carrying a label]

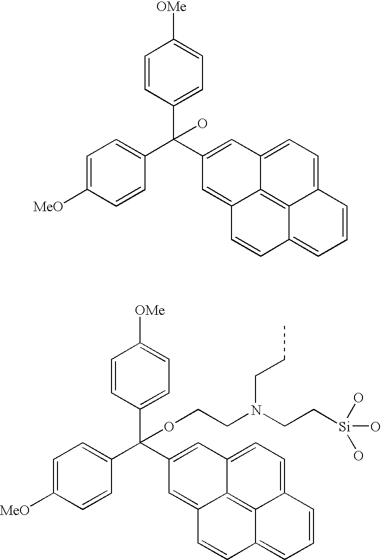

Aside from fluorescent dyes such as pyrenes, coumarines, rhodamines, cyanines, for example, nano-particles and other fluorescent labels may also be considered.

1. A method for testing functionalized groups on a surface,
(a) providing a support comprising a surface,
(b) coating at least part of the surface of said support with first functional coating molecules, using first coating molecules which carry a reversibly bound label,
(c) detecting the existence and/or density or/and distribution of the labels on the surface,
(d) removing the labels and
(e) where appropriate, coating the support with further functional coating molecules, which react with functional groups of said first coating molecules, said functional groups having preferably been uncovered due to removal of the labeling groups.

2. The method as claimed in claim 1 for preparing supports for biochips, gene chips and microarrays.

3. The method as claimed in claim 1, wherein the further functional coating molecules are selected from:
(a) nucleic acids or nucleic acid analogs or building blocks for the synthesis thereof,
(b) peptides or proteins or building blocks for the synthesis thereof,
(c) carbohydrates or building blocks for the synthesis thereof,
(d) compounds of combinatorial chemistry or building blocks for the synthesis thereof, and
(e) linker molecules or building blocks for the synthesis thereof.

4. A reagent for carrying out the method as claimed in claim 1, containing at least one first functional coating molecule having a reversibly bound label and at least one further functional coating molecule capable of reacting with functional groups of said first coating molecule, said functional groups having preferably been uncovered due to removal of the labeling groups.
